(19) United States
(12) Patent Application Publication
(10) Pub. No.: US 2003/0130275 A1
(43) Pub. Date: Jul. 10, 2003

(54) Sulfonamide compounds with pharmaceutical activity

(75) Inventor: Peter John Lovell, Harlow (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/305,450

(22) Filed: Nov. 27, 2002

Related U.S. Application Data
(63) Continuation of application No. 09/979,472, filed on Nov. 14, 2001, now abandoned.
Application No. 09/979,472 is a 371 of international application No. PCT/EP00/04893, filed on May 25, 2000.

(30) Foreign Application Priority Data
Jun. 1, 1999 (GB) 9912701.1

(51) Int. Cl. A61K 31/541; A61K 31/5377; A61K 31/496; A61K 31/454; A61K 31/4439; C07D 417/14; C07D 413/14; C 07D 4 3/02

(52) U.S. Cl. 514/227.8; 544/60; 544/121; 544/129; 544/360; 544/372; 546/187; 546/207; 546/276.4; 514/235.5; 514/235.8; 514/253.09; 514/253.12; 514/254.01; 514/326; 514/343

(57) ABSTRACT

The invention relates to novel sulphonamide compounds having 5-HT₇ antagonist activity, processes for their preparation, to compositions containing them and to their use in the treatment of CNS and other disorders.

This invention relates to compounds having pharmacological activity, processes for their preparation, to compositions containing them and to their use in the treatment of CNS and other disorders.

WO 97/29097, WO 97/48681 and WO 97/49695 all disclose a series of sulphonamide derivatives that are 5-HT₇ receptor antagonists and are said to be useful in the treatment of various CNS diseases.

A structurally novel class of compounds have now been found that also possess 5-HT₇ receptor activity. The present invention therefore provides, in a first aspect, a compound of formula (I) or a pharmaceutically acceptable salt thereof:

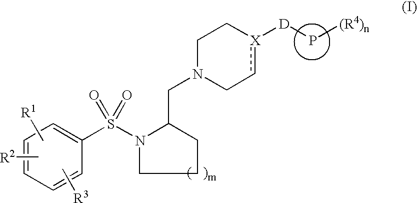

wherein:

R¹, R² and R³ are independently hydrogen, halogen, hydroxy, C₁₋₆alkyl or C₁₋₆alkoxy;

m is 1 or 2,

X is nitrogen, carbon or CH,

[symbol] is a single bond when X is nitrogen or a CH; or

[symbol] is a double bond when X is carbon;

D is a single bond, C═O, 0 or CH₂ subject to the proviso that when X is nitrogen then D is not oxygen;

P is phenyl, naphthyl, a 5 or 6 membered heteroaryl ring containing 1 to 3 heteroatoms selected from oxygen, nitrogen and sulphur, or a benzofused heteroaryl ring containing 1 to 3 heteroatoms selected from oxygen, nitrogen and sulphur;

R⁴ is C₁₋₆alkyl optionally substituted by NR⁵R⁶, aryl, arylC₁₋₆ alkyl, C₁₋₆alkoxy, C₁₋₆alkylthio, cyano, hydroxy, nitro, halogen. CF₃, C₂F₅, NR⁵R⁶, CONR⁵R⁶, NR⁵COR⁶, S(O)ₚNR⁵R⁶, CHO, OCF₃, SCF₃, COR⁷, CH₂OR⁷, CO₂R⁷ or OR⁷ where p is 0.1 or 2 and R⁵, R⁶ and R⁷ are independently hydrogen. C₁₋₆alkyl, aryl or arylC₁₋₆alkyl;

n is 0, 1, 2 or 3.

C₁₋₆alkyl groups whether alone or as part of another group may be straight chain or branched. The term ‘halogen’ is used herein to describe, unless otherwise stated, a group selected from fluorine, chlorine, bromine or iodine. The term ‘aryl’ is used herein to describe, unless otherwise stated, a group such as phenyl or naphthyl. Such aryl groups may be optionally substituted by one or more C₁₋₆alkyl or halogen.

Suitably the groups R¹, R² and R³ are independently hydrogen, halogen such as fluorine, chlorine or bromine or C₁₆alkyl such as methyl. Preferably the group R¹ is a methyl group with a meta relationship with respect to the sulphonamide linkage and both R² and R³ are hydrogen.

Preferably D is a single bond.

When P is naphthyl this is intended to denote both naphthalen-1-yl and naphthalen-2-yl groups. When P is a 5 or 6 membered heteroaryl ring suitable examples include thienyl, furyl, pyrrolyl, triazolyl, imidazolyl, oxazolyl, thiazolyl, oxadiazolyl, isothiazolyl, isoxazolyl, thiadiazolyl, pyridyl, pyrimidyl, pyrrolidinyl and pyrazinyl. When P is a benzofused heteroaryl ring suitable examples include indolyl, benzimidazolyl, benzoxazolyl, benzothiazolyl, quinolinyl and isoquinolinyl. The groups listed above can be linked to the remainder of the molecule via a carbon atom or, when present, a suitable nitrogen atom. It will be appreciated however, that when D is O then the heteroaryl or benzofused heteroaryl ring must be linked to the rest of the molecule via a carbon atom. Preferably P is phenyl, naphthyl, benzimidazol-2-yl, 2-oxo-2,3-dihydrobenzimidazol-1-yl, indol-3-yl, benzoxazol-2-yl and benzothiazol-2-yl.

Preferred examples of R⁴ groups include halogen such as fluorine or chlorine, C₁₋₆alkyl optionally substituted by NR⁵R⁶ such as methyl, hydroxy, CF₃, C₁₋₆alkoxy such as methoxy or groups COR⁷ or CO₂R⁷ in which R⁷ is methyl. When n is 2 or more the groups R⁴ can be the same or different. Preferably n is 0 or 1.

Particularly preferred compounds of the invention include:

(R)-2-(-(1-(Toluene-3-sulfonyl)-pyrrolidin-2-ylmethyl)-piperidin-4-yl)-1H-benzimidazole,

(S)-2-(1-(1-(Toluene-3-sulfonyl)-pyrrolidin-2-ylmethyl)-piperidin-4-yl)-1H-benzimidazole,

(RS)-2-(1-(1-(Toluene-3-sulfonyl)-piperidin-2-ylmethyl)-piperidin-4-y)-1H-benzimidazole,

(R)-5-Fluoro-2-(1-(1-(toluene-3-sulfonyl)-pyrrolidin-2-ylmethyl)-piperidin-4-yl)-1H-benzimidazole,

(S)-5-Fluoro-2-(1-(1-(toluene-3-sulfonyl)-pyrrolidin-2-ylmethyl)-piperidin-4-yl)-1H-benzimidazole,

(RS)-5-Fluoro-2-(1-(1-(toluene-3-sulfonyl)-piperidin-2-ylmethyl)-piperidin-4-yl)-1H-benzimidazole,

(R)-2-(1-(1-(Toluene-3-sulfonyl)-pyrrolidin-2-ylmethyl)-piperidin-4-yl)-1H-benzoxazole,

(S)-2-(1-(1-(Toluene-3-sulfonyl)-pyrrolidin-2-ylmethyl)-piperidin-4-yl)-1H-benzoxazole,

(RS)-2-(1-(1-(Toluene-3-sulfonyl)-piperidin-2-ylmethyl)-piperidin-4-yl)-1H-benzoxazole,

(R)-2-(4-(1-(Toluene-3-sulfonyl)-pyrrolidin-2-ylmethyl)-piperazin-1-yl)-1H-benzimidazole,

(S)-2-(4-(1-(Toluene-3-sulfonyl)-pyrrolidin-2-ylmethyl)-piperazin-1-yl)-1H-benzimidazole,

(RS)-2-(4-(1-(Toluene-3-sulfonyl)-piperidin-2-ylmethyl)-piperazin-1-yl)-1H-benzimidazole,

(R)-1-(2-Methoxy-phenyl)-4-(1-(toluene-3-sulfonyl)-pyrrolidin-2-ylmethyl)-piperazine,

(S)-1-(2-Methoxy-phenyl)-4-(1-(toluene-3-sulfonyl)-pyrrolidin-2-ylmethyl)-piperazine,

(RS)-1-(2-Methoxy-phenyl)-4-(1-(toluene-3-sulfonyl)-piperidin-2-ylmethyl)-piperazine,

(R)-3-(1-(1-(Toluene-3-sulfonyl)-pyrrolidin-2-ylmethyl)-piperidin-4-yl)-1H-indole,

(S)-3-(1-(1-(Toluene-3-sulfonyl)-pyrrolidin-2-ylmethyl)-piperidin-4-yl)-1H-indole,

(RS)-3-(1-(1-(Toluene-3-sulfonyl)-piperidin-2-ylmethyl)-piperidin-4-yl)-1H-indole or a pharmaceutically acceptable salt thereof.

The compounds of the formula (I) can form acid addition salts with acids, such as conventional pharmaceutically acceptable acids, for example maleic, hydrochloric, hydrobromic, phosphoric, acetic, fumaric, salicylic, citric, lactic, mandelic, tartaric and methanesulphonic.

Compounds of formula (I) may also form solvates such as hydrates, and the invention also extends to these forms. When referred to herein, it is understood that the term ‘compound of formula (I)’ also includes these forms.

Certain compounds of formula (I) are capable of existing in stereoisomeric forms including diastereomers and enantiomers and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof

The present invention also provides, in a further aspect, a process for the preparation of a compound of formula (I) or a pharmaceutically acceptable salt thereof, which process comprises coupling a compound of formula (II):

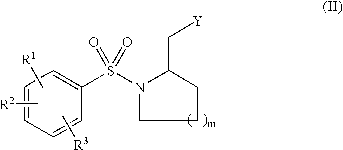

in which R¹, R², R³ and m are as defined in formula (I) and Y is a leaving group with a compound of formula (III):

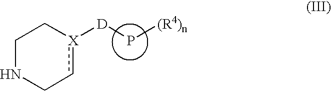

in which [symbol] , X, D, P, n and R⁴ are as defined in formula (I);

and optionally thereafter if appropriate:

removing any protecting groups;

forming a pharmaceutically acceptable salt.

Suitable leaving groups Y include halogen (particularly chloro) and OSO₂Ar groups such as tosylate. The reaction of a compounds of formulae (II) and (III) is preferably carried out in a solvent such as dichloromethane or acetonitrile optionally in the presence of sodium iodide and a base such as potassium carbonate.

Those skilled in the art will appreciate that it may be necessary to protect certain groups. Suitable protecting groups and methods for their attachment and removal are conventional in the art of organic chemistry, such as those described in Greene T. W. ‘Protective groups in organic synthesis’ New York, Wiley (1981).

Compounds of formulae (II) and (III) are described herein, are commercially available or may be prepared according to known methods or analogous to known methods.

Pharmaceutically acceptable salts may be prepared conventionally by reaction with the appropriate acid or acid derivative.

Compounds of formula (I) and their pharmaceutically acceptable salts have 5-HT₇ receptor antagonist activity and are believed to be of potential use for the treatment or prophylaxis of certain CNS disorders such as anxiety, depression, sleep disorders (including disturbances of Circadian rhythms), migraine, Parkinson's disease, schizophrenia, pain, appetite disorders and other indications such as inflammation, spastic colon, renal disorders, hypotension, cardiovascular shock, disorders associated with neuronal degeneration resulting from ischaemic events such as stroke, septic shock and gastrointestinal diseases such as IBS (irritable bowel syndrome).

Thus, the invention also provides a compound of formula (I) or a pharmaceutically acceptable salt thereof, for use as a therapeutic substance, in particular in the treatment or prophylaxis of the above disorders, particularly depression and/or sleep disorders.

The invention further provides a method of treatment or prophylaxis of the above disorders, in mammals including humans, which comprises administering to the sufferer a therapeutically effective amount of a compound of formula (I) or a pharmaceutically acceptable salt thereof. In particular the invention provides a compound of formula (I) or a pharmaceutically acceptable salt thereof or a solvate thereof for use in the treatment or prophylaxis of depression and/or sleep disorders.

The present invention also provides a pharmaceutical composition, which comprises a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration fluid unit dosage forms are prepared utilising a compound of the invention or pharmaceutically acceptable salt thereof and a sterile vehicle. The compound, depending on the vehicle and concentration used can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 20.0 mg, for example 0.2 to 5 mg; and such unit doses may be administered more than once a day, for example two or three a day, so that the total daily dosage is in the range of about 0.5 to 100 mg; and such therapy may extend for a number of weeks or months.

When administered in accordance with the invention, no unacceptable toxicological effects are expected with the compounds of the invention.

The following Descriptions and Examples illustrate the preparation of the compounds of the invention.

Description 1

(R)-Toluene-3-sulfonic acid 1-(toluene-3-sulfonyl)-pyrrolidin-2-yl methyl ester (D1)

Diisopropylethylamine (8.6 mL, 49 mmol) and toluene-3-sulfonyl chloride (8.3 g, 44 mmol) were added to a solution of D-prolinol (2.0 g, 20 mmol) in dichloromethane (100 ml). The resulting solution was heated to reflux for 15 hours. Upon cooling, the solution was added to 100 mL of saturated aqueous sodium bicarbonate. The organic phase was separated, dried over sodium sulphate and concentrated in vacuo. The residue was purified by silica gel chromatography to afford the title compound (1.6 g, 19%).

¹H NMR (250 MHz, CDCl₃) 7.75 (2H, m), 7.59 (2H, m), 7.48 (2H, m), 7.40 (2H, m), 4.26 (1H, dd, 9.9, 3.4 Hz), 4.00 (1H, dd. 9.9, 7.9 Hz), 3.77 (1H, m), 3.40 (1H, m), 3.08 (1H, m), 2.47 (3H, s), 2.43 (3H, s), 1.89-1.55 (4H, m). MS m/e 410 (MH⁺)

Description 2

(S)-Toluene-3-sulfonic acid 1-(toluene-3-sulfonyl)-pyrrolidin-2-yl methyl Ester (D2)

The title compound was prepared L-prolinol using the method described in Description 1.

1H NMR (250 MHz, CDCl₃) 7.75 (2H, m), 7.59 (2H, m), 7.48 (2H, m), 7.40 (2H, m), 4.26 (1H, dd, 9.9, 3.4 Hz), 4.00 (1H, dd, 9.9, 7.9 Hz), 3.77 (1H, m), 3.40 (1H, m), 3.08 (1H, m), 2.47 (3H, s), 2.43 (3H, s), 1.89-1.55 (4H, m). MS m/e 410 (MH⁺).

Description 3

(RS)-Toluene-3-sulfonic Acid 1-(toluene-3-sulfonyl)-piperidin-2-yl methyl Ester (D3)

The title compound was prepared piperidine-2-methanol using the method described in Description 1.

1H NMR (CDCl₃) 7.66-7.63 (4H, m), 7.46 (2H, m), 7.35 (2H, m), 4.26 (1H, m), 4.11 (2H, m), 3.72 (1H, brd, 13.7 Hz), 2.83 (1H, m), 2.45 (3H, s), 2.42 (3H, s), 1.70 (1H, m), 1.60-1.31 (5H, m). MS m/e 424 (MH⁺).

Description 4

4-(Benzimidazol-2-yl)piperidine (D4)

A mixture of 4-piperidine carboxylic acid (5.30 g, 40 mmol), 1,2-diaminobenzene (4.32 g, 40 mmol) and polyphosphoric acid (40 g) were heated to 190° C. for 14 hours, cooled, diluted with water (150 ml) and basified with 50% KOH to pH 8. The solution was cooled in an ice/salt bath to give a precipitate which was collected by filtration and washed with water. The solid was dried in vacuo to afford the title compound (8.0 g, 100%).

1H NMR (CDCl₃) 7.48 (2H, m), 7.09 (2H, m), 3.04 (2H, m), 2.92 (2H, m), 2.60 (2H, m), 2.55 (1H, m), 1.95 (2H, m), 1.71 (2H, m). MS m/e 202 (MH⁺).

Description 5

4-(5-fluorobenzimidazol-2-yl)piperidine (D5)

The title compound was prepared from isonipecotic acid and 2,3 diamino fluorobenzene using the method described in Description 4. MS m/e 220 (MH⁺).

Description 6

4-(Benzoxazol-2-yl)piperidine (D6)

The title compound was prepared from isonipecotic acid and 2-aminophenol using the method described in Description 4. MS m/e 203 (MH⁺).

Description 7

1H-Benzimidazol-2-yl piperazine (D7)

The title compound can be prepared according to procedures described in J. Med. Chem., 1997, 40, 583-593.

Description 8

1H-Indol-3-yl Piperidine (D8)

The title compound can be prepared according to procedures described in J. Med. Chem., 1992, 35(26), 4813.

### EXAMPLE 1

(R)-2-(1-(1-(Toluene-3-sulfonyl)-pyrrolidin-2-ylmethyl)-piperidin-4-yl)-1H-benzimidazole (E1)

2-(Piperidin-4-yl)-1H-benzimidazole (109 mg, 0.54 mmol) was added to a mixture of (R)-toluene-3-sulfonic acid 1-(toluene-3-sulfonyl)-pyrrolidin-2-yl methyl ester (D1) (220 mg, 0.54 mmol), potassium carbonate (150 mg, 1.1 mmol) and sodium iodide (5 mg) in acetonitile (10 mL) under argon. The reaction mixture was heated to reflux for 12 hours. Upon cooling, the solvent was removed in vacuo and the residue partitioned between dichloromethane and saturated aqueous sodium bicarbonate. The organic phase was dried over sodium sulphate and concentrated in vacuo. Chromatography on silica gel afforded the title compound.

1H NMR (CDCl₃) 7.65 (2H, m), 7.41 (2H, m), 7.22 (4H, m), 3.76 (1H, m), 3.44 (1H, m), 3.15-2.91 (4H, m), 2.71 (1H, dd, 12.7, 3.9 Hz), 2.44 (3H, s), 2.38-2.20 (4H, m), 2.17-1.75 (6H, m). MS m/e 439 (MH⁺). Examples E2-E12 shown in Table 1 were prepared using the procedure described in Example 1 using either D1 or D2 and a 4-substituted piperidine or piperazine. Such reagents are either commercially available or can be prepared by methods described above.

TABLE 1

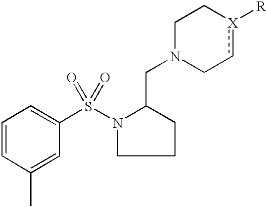

| Ex. | Stereochem. | X | R | MH⁺ |
|---|---|---|---|---|
| E2 | R | CH | 5-Fluoro-1H-benzimidazol-2-yl | 457 |
| E3 | R | CH | Benzoxazol-2-yl | 440 |
| E4 | R | N | 1H-benzimidazol-2-yl | 440 |
| E5 | R | N | 2-methoxyphenyl | 430 |
| E6 | R | CH | 1H-Indol-3-yl | 438 |
| E7 | S | CH | 1H-benzimidazol-2-yl | 439 |
| E8 | S | CH | 5-Fluoro-1H-benzimidazol-2-yl | 457 |
| E9 | S | CH | Benzoxazol-2-yl | 440 |
| E10 | S | N | 1H-benzimidazol-2-yl | 440 |
| E11 | S | N | 2-methoxyphenyl | 430 |
| E12 | S | CH | 1H-Indol-3-yl | 438 |

Examples E13-E18 shown in Table 2 were prepared using the procedure described in Example 1 using D3 and a 4-substituted piperidine or piperazine. Such reagents are either commercially available or can be prepared by methods described above.

TABLE 2

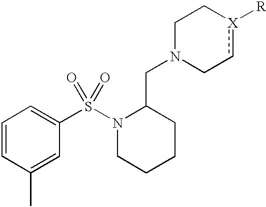

| Ex | Stereochem. | X | R | MH⁺ |
|---|---|---|---|---|
| E13 | RS | CH | 1H-benzimidazol-2-yl | 453 |
| E14 | RS | CH | 5-Fluoro-1H-benzimidazol-2-yl | 471 |
| E15 | RS | CH | Benzoxazol-2-yl | 454 |
| E16 | RS | N | 1H-benzimidazol-2-yl | 454 |
| E17 | RS | N | 2-methoxyphenyl | 444 |
| E18 | RS | CH | 1H-Indol-3-yl | 452 |

Pharmacological Data

[³H]-5-Carboxamidotryptamine Binding to Human 5-HT₇ Receptor Clones Expressed in 293 Cells in vitro.

The affinity of the compounds of this invention for the 5-HT₇ receptor binding site can be determined by methods described in WO 97/29097.

All compounds tested had a pKi in the range 6.0-7.9.

1. A compound of formula (I) or a pharmaceutically acceptable salt thereof: 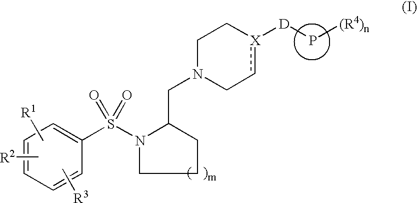
wherein:
R¹, R² and R³ are independently hydrogen, halogen, hydroxy, C₁₋₆alkyl or C₁₋₆alkoxy;
m is 1 or 2;
X is nitrogen, carbon or a CH, [symbol]
is a single bond when X is nitrogen or CH; or [symbol]
is a double bond when X is carbon;
D is a single bond, C═O, 0 or CH₂ subject to the proviso that when X is nitrogen then D is not oxygen;
P is phenyl, naphthyl, a 5 or 6 membered heteroaryl ring containing 1 to 3 heteroatoms selected from oxygen, nitrogen and sulphur, or a benzofused heteroaryl ring containing 1 to 3 heteroatoms selected from oxygen, nitrogen and sulphur;
R⁴ is C₁₋₆alkyl optionally substituted by NR⁵R⁶, aryl, arylC₁₋₆ alkyl, C₁₋₆alkoxy, C₁₋₆alkylthio, cyano, hydroxy, nitro, halogen, CF₃, C₂F₅, NR⁵R⁶, CONR⁵R⁶, NR⁵COR⁶, S(O)ₚNR⁵R⁶, CHO, OCF₃, SCF₃, COR⁷, CH₂OR⁷, CO₂R⁷ or OR⁷ where p is 0, 1 or 2 and R⁵, R⁶ and R⁷ are independently hydrogen, C₁₋₆alkyl, aryl or arylC₁₋₆alkyl;
n is 0, 1, 2 or 3.

2. A compound according to claim 1 in which R¹ is a methyl group with a meta relationship with respect to the sulphonamide linkage and both R² and R³ are hydrogen.

3. A compound according to claim 1 or claim 2 in which D is a single bond.

4. A compound according to any one of the preceding claims in which P is benzimidazole.

5. A compound according to claim 1 which is
(R)-2-(1-(1-(Toluene-3-sulfonyl)-pyrrolidin-2-ylmethyl)-piperidin-4-yl)-1H-benzimidazole,
(S)-2-(1-(1-(Toluene-3-sulfonyl)-pyrrolidin-2-ylmethyl)-piperidin-4-yl)-1H-benzimidazole,
(RS)-2-(1-(1-(Toluene-3-sulfonyl)-piperidin-2-ylmethyl)-piperidin-4-yl)-1H-benzimidazole,
(R)-5-Fluoro-2-(1-(1-(toluene-3-sulfonyl)-pyrrolidin-2-ylmethyl)-piperidin-4-yl)-1H-benzimidazole,
(S)-5-Fluoro-2-(1-(1-(toluene-3-sulfonyl)-pyrrolidin-2-ylmethyl)-piperidin-4-yl)-1H-benzimidazole,
(RS)-5-Fluoro-2-(1-(1-(toluene-3-sulfonyl)-piperidin-2-ylmethyl)-piperidin-4-yl)-1H-benzimidazole,
(R)-2-(1-(1-(Toluene-3-sulfonyl)-pyrrolidin-2-ylmethyl)-piperidin-4-yl)-1H-benzoxazole,
(S)-2-(1-(1-(Toluene-3-sulfonyl)-pyrrolidin-2-ylmethyl)-piperidin-4-yl)-1H-benzoxazole,
(RS)-2-(1-(1-(Toluene-3-sulfonyl)-piperidin-2-ylmethyl)-piperidin-4-yl)-1H-benzoxazole,
(R)-2-(4-(1-(Toluene-3-sulfonyl)-pyrrolidin-2-ylmethyl)-piperazin-1-yl)-1H-benzimidazole,
(S)-2-(4-(1-(Toluene-3-sulfonyl)-pyrrolidin-2-ylmethyl)-piperazin-1-yl)-1H-benzimidazole,
(RS)-2-(4-(1-(Toluene-3-sulfonyl)-piperidin-2-ylmethyl)-piperazin-1-yl)-1H-benzimidazole,
(R)-1-(2-Methoxy-phenyl)-4-(1-(toluene-3-sulfonyl)-pyrrolidin-2-ylmethyl)-piperazine,
(S)-1-(2-Methoxy-phenyl)-4-(1-(toluene-3-sulfonyl)-pyrrolidin-2-ylmethyl)-piperazine,
(RS)-1-(2-Methoxy-phenyl)-4-(1-(toluene-3-sulfonyl)-piperidin-2-ylmethyl)-piperazine,
(R)-3-(1-(1-(Toluene-3-sulfonyl)-pyrrolidin-2-ylmethyl)-piperidin-4-yl)-1H-indole,
(S)-3-(1-(1-(Toluene-3-sulfonyl)-pyrrolidin-2-ylmethyl)-piperidin-4-yl)-1H-indole,
(RS)-3-(1-(1-(Toluene-3-sulfonyl)-piperidin-2-ylmethyl)-piperidin-4-yl)-1H-indole, or a pharmaceutically acceptable salt thereof.

6. A process for the preparation of a compound of formula (I) or a pharmaceutically acceptable salt thereof, which process comprises coupling a compound of formula (II): 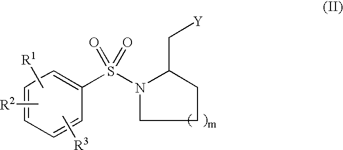
in which R¹, R², R³ and m are as defined in formula (I) and Y is a leaving group with a compound of formula (III) 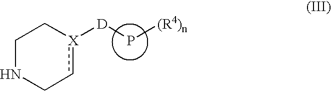
in which [symbol] , X, D, P, n and R⁴ are as defined in formula (1);
and optionally thereafter if appropriate:
removing any protecting groups;
forming a pharmaceutically acceptable salt.

7. A compound according to any one of claims 1 to 5 for use in therapy.

8. A compound according to any one of claims 1 to 5 for use in the treatment of CNS disorders.

9. A pharmaceutical composition which comprises a compound according to any one of claims 1 to 5 and a pharmaceutically acceptable carrier or excipient.

10. The use of a compound according to any one of claims 1 to 5 in the manufacture of a medicament for use in the treatment of depression and/or sleep disorders.
